(19) United States
(12) Patent Application Publication
(10) Pub. No.: US 2024/0335316 A1
(43) Pub. Date: Oct. 10, 2024

(54) JOHN BAG

(71) Applicant: Patricia C. Sands, Arlington, VA (US)

(72) Inventor: Patricia C. Sands, Arlington, VA (US)

(21) Appl. No.: 18/628,660

(22) Filed: Apr. 5, 2024

Related U.S. Application Data
(60) Provisional application No. 63/577,162, filed on Apr. 5, 2023.

(51) Int. Cl. A61F 5/453; A47K 11/12

(52) U.S. Cl. CPC A61F 5/453; A47K 11/12

(57) ABSTRACT

A John Bag is formed from a flexible fabric, has a closed end, an open end, and top and bottom panels, as a privacy cloth which is held in place by an adjustable band when not in use. When in use, the privacy cloth is removed from the adjustable band and pulled over the open end and extends to completely cover a user's private parts. A urinal is disposed within the John Bag, the urinal having an opening which is exposed at the open end of the John Bag. There is an optional stretch internal insert to keep the variety of bottle shapes stable and secure, making it tip and spill resistant. The John bag has an optional pocket for a scent package or to use as a freshener.

### CROSS REFERENCE TO RELATED APPLICATIONS

This application claims the priority of Provisional Application No. 63/577,163 filed Apr. 5, 2023, titled “The John Bag”, inventor Patricia Sands. The entire disclosure of this provisional patent application is hereby incorporated by reference thereto, in its entirety.

### STATEMENT REGARDING FEDERALLY SPONSORED RESEARCH OR DEVELOPMENT

Not applicable.

### FIELD OF THE INVENTION

The present invention relates to receptacles, bags, and enclosures for receiving and holding urinal bottles.

### BACKGROUND OF THE INVENTION

It is a problem in the art to provide a portable urinal for use by male individuals, which also affords privacy and convenience.

Portable urinals are well known, and include bottles and containers. However, it is a problem to ensure modesty and privacy when using such a portable urinal.

### SUMMARY OF THE INVENTION

From the foregoing, it is seen that it is a problem in the art to provide a device meeting the above requirements. According to the present invention, a device is provided which meets the aforementioned requirements and needs in the prior art. Specifically, the device according to the present invention provides a device to ensure privacy for a male user of a portable urinal.

The device of the present invention provides a John Bag, which is preferably formed from a flexible fabric such as nylon, polyester, and the like. The material Spandex® can also be used. The John Bag has a closed end, an open end, and top and bottom panels. A privacy cloth is provided that is attached to an exterior portion of the John Bag, and is held in place by an adjustable band when not in use. When in use, the privacy cloth is removed from the adjustable band and pulled over the open end and extending to completely cover the male user's private parts. A urinal is disposed within the John Bag, the urinal having an opening which is exposed at the open end of the John Bag.

The John Bag is custom designed to fit a plastic male portable urinal with an opening for the spout.

The bag has a top with a slit or hole to receive the spout of the urinal to protrude through, and a privacy cover cloth that is soon to the back body or optionally can be removed from the bag body.

There is an optional stretch internal insert to keep the variety of bottle shapes stable and secure, making it tip and spill resistant.

The bag has an adjustable band or strap to tighten the fit of various size urinal products so they are secure in the bag and do not spill. The bag will have the industry standard handle to carry, or to hang on its chair or a wheelchair.

The John bag has a combination of several materials with unique features. The bag is preferably made from a waterproof and/or water resistant material with a custom slit for use of a contained product. The cover will be made of folded fabric that will expand to cover the entire lap. The John bag will be washable and designed for long-term care.

The John bag has an optional pocket for a scent package or to use as a freshener.

Other objects and advantages of the present invention will be more readily apparent from the following detailed description when read in conjunction with the accompanying drawings.

### BRIEF DESCRIPTION OF THE DRAWINGS

FIG. 1 is a perspective view of a John Bag according to the present invention from the right side, when in use by a user U shown in phantom outline.

FIG. 2 is a perspective view from the front and right side of a Bag according to the present invention, having a privacy cloth raised to reveal a front and right side partial view of a Urinal shown in phantom outline, the Urinal having an opening.

FIG. 3 is a perspective view of the bag as seen from the bottom right rear of the bag of FIG. 1, with the privacy cloth wrapped around to cover an opening in the bag, the opening being on the right side in this view (not shown in this view).

FIG. 4 is a perspective view of the bag as seen from the top left rear, corresponding to the bag of FIG. 3, and with the privacy cloth secured by the adjustable band.

FIG. 5 is a perspective view of the bag as seen from the bottom left rear, showing the privacy cloth in an open configuration.

FIG. 6 is a perspective view of the bag as seen from the left, and upside down as compared with the position of the John Bag of FIG. 1, showing a urinal bottle B in phantom outline partially removed from the bag, the bottle B being upside down and the privacy cloth being disposed away from the bottle B.

FIG. 7 is a perspective view of the bag of FIG. 1 as seen from the right side, with the privacy cloth 10 being lifted to reveal an opening of the Bottle B, and showing the opening in the bag.

FIG. 8 is a perspective view as seen from the right of the bag, corresponding to FIG. 7, with the privacy cover being lowered over the opening of the Bottle B of FIG. 7.

FIG. 9 is a perspective view of the bag as seen from above, with the front side of the bag disposed on the left of this figure and the rear portion of the bag being disposed on the right of this figure; this view showing a configuration wherein the privacy cloth is disposed pulled away from the opening of the bag, the privacy cloth extending past the bottom end of the bag.

FIG. 10 is a perspective view of the bag 100 of FIG. 9 as seen from the bottom, showing the adjustable band, and also showing a first pocket and a second pocket; the privacy cloth being shown in a configuration wherein the privacy cloth is disposed pulled away from the opening of the bag and extending past the bottom end of the bag.

FIG. 11 is a perspective view of the bag in an upright position wherein the opening is disposed at the top of this view, and shows the bottom left of the bag in a closed configuration ready for carrying.

FIG. 12 is a perspective view of the bag of FIG. 11, shown in an upright position wherein the opening is disposed at the top of this view, and shows the bottom left of the bag in a closed configuration ready for carrying.

FIG. 13 is a perspective view of an insert member which is flexible and stretchable, for receiving a urinal bottle securely. A phantom outline is shown indicating schematically a body of the bag, and showing an end.

### DETAILED DESCRIPTION OF THE INVENTION

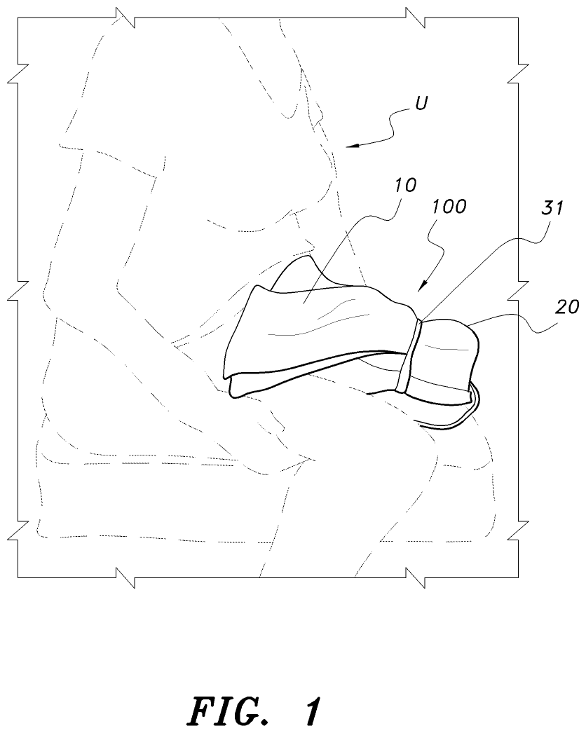

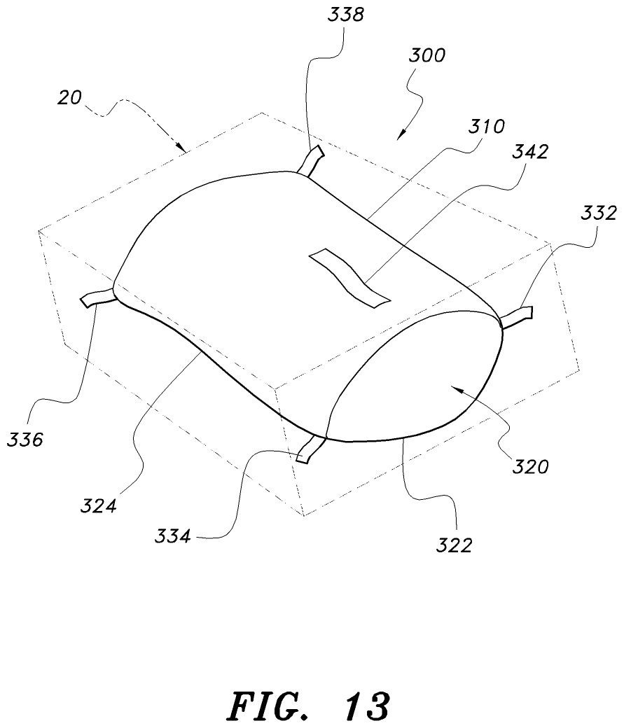

The John bag 100 (seen in FIGS. 1-5) of the present invention is a custom personal carry and cover privacy bag that is intended to be used as both a carrier bag and a cover, for when a urinal/personal care product is being used. The bag 100 is custom designed to fit a variety of male urinals with adjustable strap or band 30. The bag 100 has a slit opening for a urinal spout to protrude through the bag 100. This feature eliminates the need to take the urinal out of the bag 100 to use it. There is an optional internal stretch insert 300 (shown in FIG. 13) to hold the bottle securely. The John bag 100 has a cover cloth (privacy cloth 10) that is sewn or detachable to the body of the bag. The cloth serves as a privacy cloth cover when the user U of FIG. 1 is voiding into the urinal.

The John Bag 100 is constructed so as to give privacy that users need when using a male urinal, and who want to avoid the embarrassment of carrying or using the urinal without a cover. The John Bag 100 is both a carry bag and a discreet “in use” cover. The bag 100 has a custom slit or opening for the urinal spout to protrude through the bag 100 and a cover cloth 10 that is attached. The cloth will serve to cover the male user and his private parts while he is using the urinal.

The invention is described further below with reference to the drawing figures.

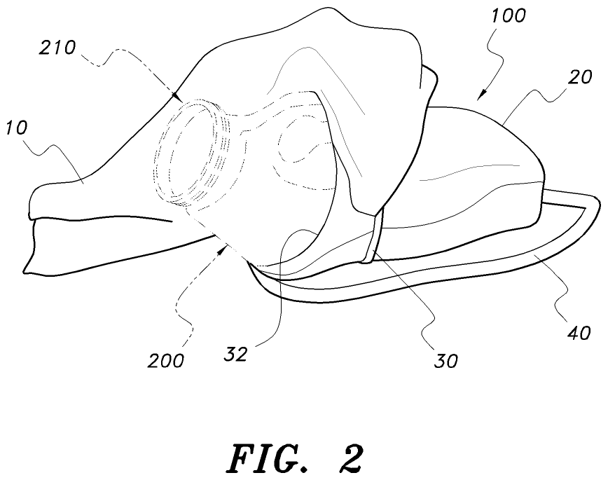

FIG. 1 is a perspective view of a John Bag 100 according to the present invention, as seen from the right side, when in use by a user U shown in phantom outline. In this view, the bag 100 has a privacy cloth 10, a flexible cloth or fabric body 20. A seam 31 is shown, where the privacy cloth 10 is attached to the fabric body 20. The bag 100 contains a urinal (not shown in this view, and is shown in FIG. 2), wherein the user U can eliminate urine into the urinal in a modest manner, in which the private parts of the user U are covered by the privacy cloth 10.

FIG. 2 is a perspective view from the front and right side of the Bag 10 according to the present invention, having the privacy cloth 10 raised to reveal a front and right side partial view of a Urinal 200 shown in phantom outline, the urinal 200 having an opening 210. The urinal 200 is disposed in an opening of the bag 100 bounded by an opening edge 32.

FIG. 3 is a perspective view of the bag as seen from the bottom right rear of the bag 100 of FIG. 1, with the privacy cloth 10 being wrapped around to cover an opening in the bag, the opening being on the right side in this view, the opening itself not being shown in this view as it is covered by the privacy cloth 10. The bag 100 has an end portion 70. A handle 50 is shown, attached at opposite sides of the bag 100, for carrying purposes. The carrying handle 50 shown in this view can be the same as handle 40 of FIG. 2, or can be of a different construction or different material, different cross-section, and/or different length as compared to that of handle 40 of FIG. 2.

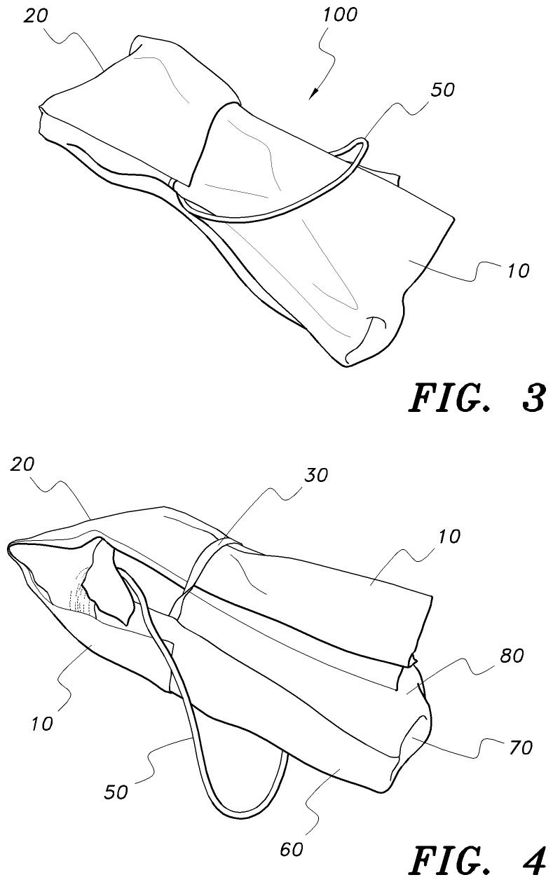

FIG. 4 is a perspective view of the bag 100 as seen from the top left rear of the bag 100, corresponding to the position shown in FIG. 3, and with the privacy cloth 10 being secured by the adjustable band 30. The bag 100 has an end portion 70, a top portion 80, and a bottom portion 60. The privacy cloth 10 is wrapped over the upper portion 80 and disposed beneath the adjustable band 30. The adjustable band 30 thereby retains the privacy cloth 10 in place. In this view, there is no urinal inside the bag 100, so that the privacy cloth 10 can be shown clearly in a configuration wherein it is wrapped around over the top portion 80, and extending all the way around where it is connected to the bottom portion 60. The end 20 near the opening of the bag is also shown in this view.

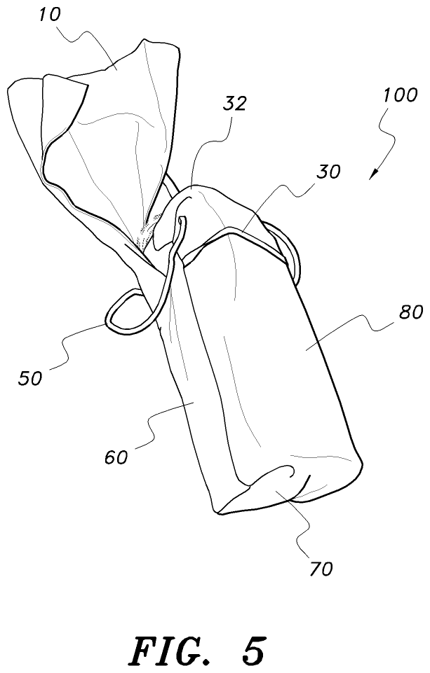

FIG. 5 is a perspective view of the bag 100 as seen from the bottom left rear, showing the privacy cloth 10 in an open configuration. This view shows the privacy cloth 10 extended away from the body portion of the bag 100, wherein the body portion is formed by the top portion 80, the bottom portion 60, and the end portion 70.

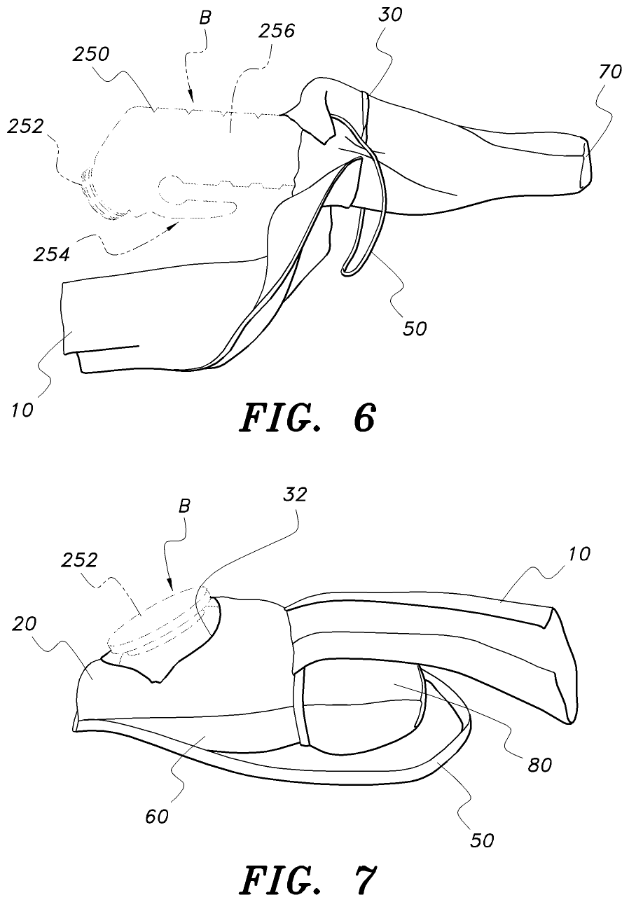

FIG. 6 is a perspective view of the bag as seen from the left, and upside down as compared with the position of the John Bag of FIG. 1, showing a urinal bottle B in phantom outline partially removed from the bag, the bottle B being upside down and the privacy cloth being disposed away from the bottle B. The bottle B may be different than the bottle 200 shown in FIG. 2, or may be the same or similar, and any type of bottle being usable in the present invention, the bottle having the approximate shape of the bag 100, or conversely the bag 100 can be sized and dimensioned to fit other types of bottles having different dimensions than those shown in FIG. 6. The bottle B of this view has an opening 252, a handle 254, a bottom 250, and a side wall 256.

FIG. 7 is a perspective view of the bag 100 of FIG. 1 as seen from the right side, with the privacy cloth 10 being lifted to reveal an opening 252 of the Bottle B, and showing the opening 32 in the bag 100. A cap may be provided for the opening 252, as is usual for urinal bottles.

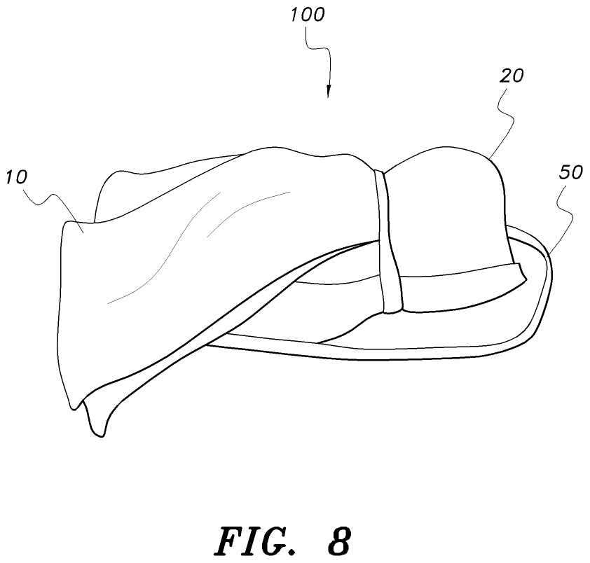

FIG. 8 is a perspective view as seen from the right of the bag 100, corresponding to FIG. 7, with the privacy cover 10 being lowered over the opening (not shown in this figure, shown in FIG. 7) of the Bottle B of FIG. 7.

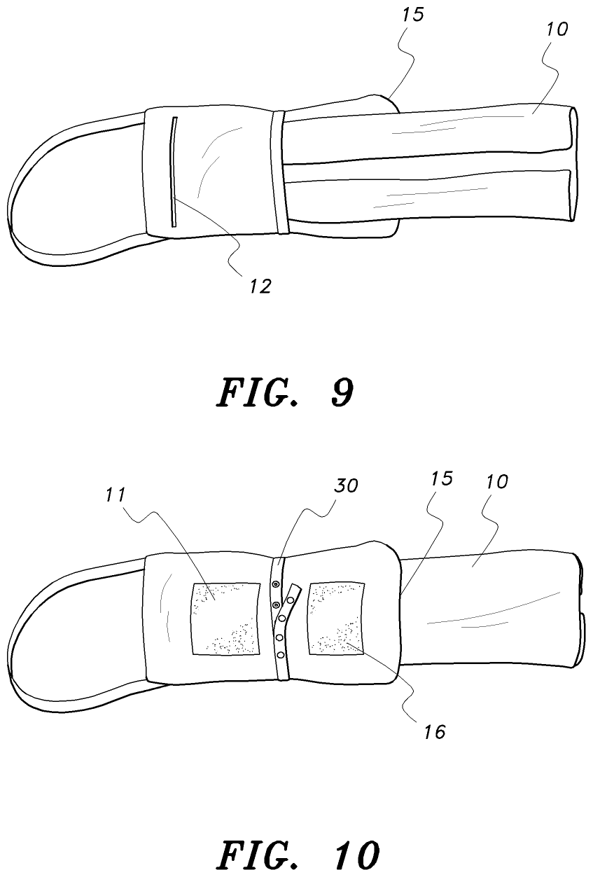

FIG. 9 is a perspective view of the bag 100 as seen from above, with the front side of the bag 100 disposed on the left of this figure and the rear portion of the bag 100 being disposed on the right of this figure; this view showing a configuration wherein the privacy cloth 10 is disposed pulled away from the opening of the bag 100, the privacy cloth 10 extending past the bottom end of the bag 100. An opening 12 is shown in this view, which is slit-like and allows insertion of a urinal bottle into the bag 100.

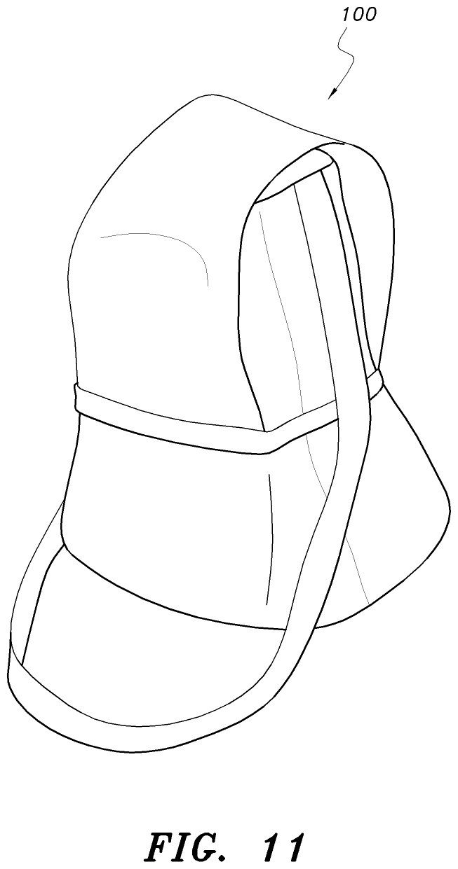

FIG. 10 is a perspective view of the bag 100 of FIG. 9 as seen from the bottom, showing the adjustable band 30, and also showing a first pocket 11 and a second pocket 16; the privacy cloth 10 being shown in a configuration wherein the privacy cloth 10 is disposed pulled away from the opening of the bag 100 and extending past the bottom end of the bag 100.

The privacy cloth 10 has optional Velcro® pads or strips attached near its free end, for sticking and/or attaching to the material forming the body of the bag 100.

FIG. 11 is a perspective view of the bag 100 in an upright position wherein the opening is disposed at the top of this view, and shows the bottom left of the bag in a closed configuration ready for carrying.

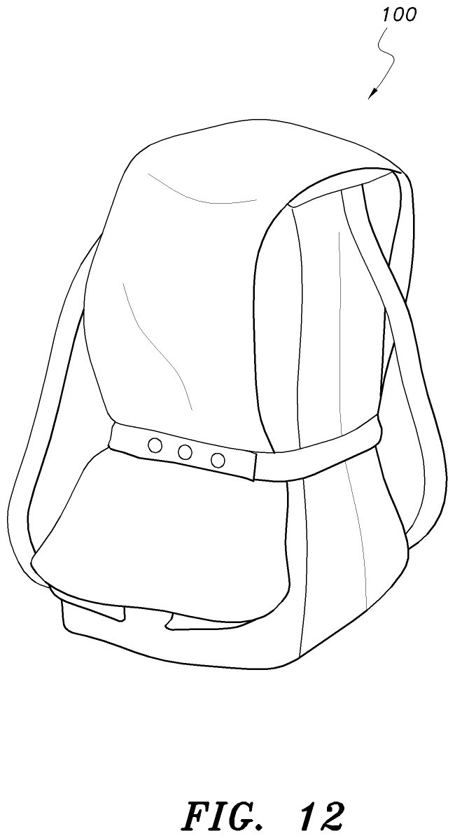

FIG. 12 is a perspective view of the bag 100 of FIG. 11, shown in an upright position wherein the opening is disposed at the top of this view, and shows the bottom left of the bag in a closed configuration ready for carrying.

FIG. 13 is a perspective view of an insert member 300 which is flexible and stretchable, for receiving a urinal bottle securely. A phantom outline is shown indicating schematically a body of the bag 100, and showing an end. 20. The insert body 300 has a bottom surface 322, a closed bottom having an end surface 320, and a left side 324 and a right side 310. The insert member 300 is open at a side opposite from the bottom 320, near the arrow 20 which indicates the opening of the bag 100. There are respective Velcro® strips 334, 336, 338, 342, and 332, schematically shown, for attachment to the interior of the body portion of the bag 100.

The invention being thus described, it will be evident that the same may be varied in many ways by a routineer in the applicable arts. Such variations are not to be regarded as a departure from the spirit and scope of the invention and all such modifications are intended to be included within the scope of the claims.

What is claimed is:

1. A device for privacy of a user of a urinal bottle which has a urinal opening, comprising:
a body which is flexible;
said body having a closed end portion, an open end portion, a top portion, and a bottom portion; and
a privacy cloth attached to said body, having a first configuration in which the privacy cloth is secured to said body, and a second configuration which is a privacy configuration wherein said privacy cloth is extended over said open end portion;
wherein in use, the urinal bottle is disposed inside said body such that the opening of the urinal bottle is disposed at said open end portion, such that when the user is voiding into the urinal bottle the privacy cloth covers the opening of the urinal bottle and covers the user.

2. The device of claim 1, wherein said body is formed from a flexible fabric

3. The device of claim 2, wherein said flexible fabric is nylon, polyester, and the like.

4. The device of claim 1, further comprising an adjustable band secured to said body, for securing said privacy cloth when not in use.

5. The device of claim 1, further comprising a stretch internal insert to keep the variety of bottle shapes stable and secure, making it tip and spill resistant.

6. The device of claim 1, further comprising a pocket on said body.

7. The device of claim 1, further comprising a first pocket secured to said body, and a second pocket secured to said body.
